(19) United States
(12) Patent Application Publication
(10) Pub. No.: US 2006/0272036 A1
(43) Pub. Date: Nov. 30, 2006

(54) Ruminant mhc class-i-like fc receptors

(76) Inventors: Lennart Hammarstrom, Huddinge (SE); Imre Kacskovics, Budakeszi (HU)

Correspondence Address: BACON & THOMAS, PLLC, 625 SLATERS LANE, FOURTH FLOOR, ALEXANDRIA, VA, 22314, US

(21) Appl. No.: 10/181,951

(22) PCT Filed: Feb. 2, 2001
(86) PCT No.: PCT/SE01/00202

§ 371 Date: Aug. 2, 2002

Related U.S. Application Data
(60) Provisional application No. 60/180,130, filed on Feb. 3, 2000.

(51) Int. Cl. A01K 67/027

(52) U.S. Cl. 800/7; 800/14; 800/15; 800/16

(57) ABSTRACT

Immunoglobulin G (IgG) transporting ruminant Fc receptor (FcRn) α-chain DNA molecules, especially those of cow, dromedary and sheep (SEQ ID NO:1, SEQ ID NO:2, SEQ ID NO:3) are disclosed. Protein expressed by said FcRn α-chain DNA molecules are disclosed and include SEQ ID NO:4, SEQ ID NO:5, SEQ ID NO:6. Vectors containing the ruminant IgG transporting FcRn α-chain DNA molecules, and hosts transformed with such vectors are also included. Further, a method of producing colostrums or milk with enhanced levels of immunoglobulins or proteins fused to immunoglobulin γ-chains of FcRn interacting parts thereof is also disclosed.

The present invention relates to ruminant MHC class I-like Fc receptors, more precisely immunoglobulin G (IgG) transporting ruminant, especially bovine (cow), dromedary and sheep, Fc receptor (FcRn) α-chain DNA molecules, and proteins encoded by said DNA molecules. The invention also relates to vectors containing the DNA molecules of the invention, and hosts comprising the vectors. Additionally, the invention comprises a method of producing milk with enhanced levels of immunoglobulins or proteins fused to immunoglobulin γ-chains or FcRn interacting parts thereof.

### BACKGROUND OF THE INVENTION

The transfer of passive immunity from the mother to the calf in ruminants involves passage of immunoglobulins through the colostrum (1). Upon ingestion of the colostrum, immunoglobulins are transported across the intestinal barrier of the neonate into its blood. Whereas this intestinal passage appears to be somewhat non-specific for types of immunoglobulins, there is a high selectivity in the passage of these proteins from the maternal plasma across the mammary barrier into the colostrum (2). There is a rapid drop in the concentration of all lacteal immunoglobulins immediately postpartum and the selectivity of this transfer has led to the speculation that a specific transport mechanism across the mammary epithelial cell barrier is involved.

The protein responsible for transfer of maternal IgG in man, mouse and rat, the FcRn, consist of a heterodimer of an integral membrane glycoprotein, similar to MHC class I α-chains, and β2-microglobulin (3). IgG has been observed in transport vesicles in neonatal rat intestinal epithelium (4). Studies have shown that FcRn is also expressed in the fetal yolk sac of rats and mice (5), in adult rat hepatocytes (6) and in the human placenta (8, 9). More recently, Cianga et al. (9) have shown that the receptor is localized to the epithelial cells of the acini in mammary gland of lactating mice. They have suggested that FcRn plays a possible role in regulating IgG transfer into milk in a special manner in which FcRn recycles IgG from the mammary gland into the blood. The FcRn is expressed in a broad range of tissues and shows different binding affinity to distinct isotypes of IgG and the correlation between serum half-life, materno-fetal transfer and affinity of different rat IgG isotypes for the mouse FcRn was recently demonstrated (10).

The present invention now provides the isolation of cDNAs encoding ruminant homologues of the rat, mouse and human IgG transporting Fc receptor, FcRn, in particular such receptors in the cow, dromedary and sheep, and their use in vectors containing the DNA molecules and hosts comprising the vectors.

### SHORT DESCRIPTION OF THE INVENTION

The bovine cDNA, and deduced amino acid sequence, shows high similarity to the FcRn in other species and it consists of three extracellular domains, a hydrophobic transmembrane region, and a cytoplasmic tail. Aligning the known FcRn sequences, we noted that the bovine protein shows a three amino acid deletion compared to the rat and mouse sequences in the α1 loop. The presence of bFcRn transcripts in multiple tissues, including the mammary gland, suggests their involvement both in IgG catabolism and transcytosis. In addition, the cDNA of the full length coding region plus part of the 3′-end untranslated region, and deduced amino acid sequence, of sheep, and the cDNA of dromedary missing the first 301 nucleotides of the cDNA compare to the bovine cDNA sequence, and the deduced amino acid sequence missing the first 62 amino acids, compared to the bovine and sheep sequences, are disclosed.

Overexpression of ruminant FcRn through transient or persistent transgenesis using the FcRn α-chain DNA molecules according to the invention will, either alone or by concomitant upregulation of the expression of the corresponding β2-microglobulin gene, result in an increase in the number of functional receptors in the udder and thus enhance the transport of immunoglobulins and/or proteins fused to immunoglobulin γ-chains or FcRn interacting parts thereof containing the constant region of the heavy chain of IgG. Thus, not only will antibodies acquired through natural exposure or deliberate vaccination be transported more effectively into the colostrum/milk, but proteins tagged with the γ-chain (i.e. proteins where the encoding gene of interest has been linked to sequences encoding part or the whole heavy chain constant region gene for IgG), will also be more effectively transported into the colostrum/milk of ruminants. The latter proteins may be produced by animals transiently (such as through, but not limited to DNA vaccination) or persistantly (such as through, but not limited to “conventional” transgenesis) expressing the gene construct.

The FcRn transgenic ruminant animal will express the FcRn α-chain gene (with or without concomitant β2 microglobulin expression), and expression in the target organ can be directed by introducing the transgene(s) directly into the udder or, through appropriate gene targeting in “conventional” transgenic animals, be expressed in the udder.

### DETAILED DESCRIPTION OF THE INVENTION

The present invention is in one aspect directed to an immunoglobulin G (IgG) transporting ruminant Fc receptor (FcRn) α-chain DNA molecule, wherein the ruminant is preferably selected from the group consisting of cow, dromedary and sheep. In particular, the DNA molecule comprises a nucleotide sequence selected from the group consisting of SEQ ID NO: 1, SEQ ID NO: 2, SEQ ID NO: 3, and modified sequences of these three sequences expressing proteins with IgG transporting function.

It should be understood that the DNA molecule of the invention can be isolated and purified from biological (ruminant) sources or can be produced by genetic engineering.

The term “modified sequences of these three sequences expressing proteins with IgG transporting function” is used in the specification and claims to cover sequences that are truncated and sequences that have nucleotide mismatches, but still express proteins with IgG transporting function.

Another aspect of the invention is directed to a protein expressed by a ruminant FcRn α-chain DNA molecule, wherein the ruminant is preferably selected from the group consisting of cow, dromedary and sheep. In particular, the protein comprises an amino acid sequence selected from the group consisting of SEQ ID NO: 4, SEQ ID NO: 5, SEQ ID NO: 6, and modified sequences of these three sequences with IgG transporting function.

It should be understood that the DNA molecule of the invention can be isolated and purified from biological (ruminant) sources or can be produced by genetic engineering.

The term “modified sequences of these three sequences with IgG transporting function” is used in the specification and claims to cover sequences that are truncated and sequences that have amino acid mismatches, but still express proteins with IgG transporting function.

Yet another aspect of the invention is directed to a vector containing a ruminant IgG transporting FcRn α-chain DNA molecule according to the invention. Examples of vectors are plasmids and phages.

Still another aspect of the invention directed to a host transformed with a vector according to the invention. Examples of hosts are bacteria, yeasts, and ruminants, such as cows, camels, e.g. dromedaries, and sheep.

The ruminant FcRn α-chain DNA molecules of the invention and the proteins the invention may be used as tools in research work, and in the production of vectors of the invention.

The vectors of the invention may be used for the production of a transgenic ruminant animal or a local transgenic ruminant mother (i.e. injection into the udder).

Thus, an additional aspect of the invention is directed to a method of producing colostrums or milk with enhanced levels of immunoglobulins or proteins fused to immunoglobulin γ-chains or FcRn interacting parts thereof, comprising the steps of transferring a ruminant FcRn α-chain DNA molecule according to the invention through transient or persistent transgenesis into the corresponding ruminant animal for overexpression of a protein according to the invention, optionally at concomitant upregulation of the expression of the corresponding β2-microglobulin gene, to increase the number of functional receptors in the udder, thereby enhancing the transport of immunoglobulins and/or proteins fused to immunoglobulin γ-chains or FcRn interacting parts thereof from, or through, the udder into the colostrums or milk.

Examples of proteins that can be suitably produced in the milk as fusion proteins are coagulation products, such as Factor VIII, and proteins used in medicines and food.

The invention will now be further illustrated with reference to the description of drawings, experiments, and sequence listing, but the scope of protection is not intended to be limited to the disclosed embodiments of the invention.

The invention is illustrated in detail with regard to the bovine (cow) FcRn gene as a representative example of a ruminant FcRn gene, but the cDNA sequence of sheep and a partial cDNA sequence of dromedary, and the corresponding deduced amino acid sequences, are also disclosed in the sequence listing. The FcRn genes of sheep and dromedary have been produced by use of the same principal as used for obtaining the bovine FcRn gene. In particular, the same or similar primers have been used to amplify the FcRn alpha-chain encoding gene in sheep and dromedary.

### DESCRIPTION OF THE DRAWINGS

FIG. 1. The nucleotide sequence and deduced amino acid sequence of two forms of bovine FcRn α-chain. The potential ATG start is marked by bold characters, while the segment that refers to the consensus initiation site is underlined; shaded numbers in this motif represents important residues (−3-A; +4-C) of the translation signal. The predicted NH₂-terminal after signal peptide cleavage is indicated by +1 under Ala. The hydrophobic membrane-spanning segment is marked by italic characters while the polyadenylation signal AATAAA in the 3′-UT is underlined.

The sequence data have been submitted to the NCBI Nucleotide Sequence Databases under the accession number: AF139106.

FIG. 2. Domain by domain alignment of the predicted amino acid sequences for rat, mouse, bovine and human FcRn α-chains. The N-linked glycosylation site, which is found in all the sequences is indicated by a filled triangle, while empty triangles indicate additional sites in the rat and the mouse sequences. Dashed underline indicates residues that potentially interact with the Fc. The gray bar indicates the hydrophobic transmembrane region, and the asterisk represents the stop signal in the bovine sequences. Residues in an empty box following the stop signal shows the conserved carboxyl-end of the bovine cytoplasmic domain. Consensus residues are assigned based on the number of occurrences of the character in the column, emphasizing the degree of conservation. The higher the conservation in a column the darker the background of the character. (Nicholas, K. B. and Nicholas, H. B. Jr. 1997. GeneDoc: a tool for editing and annotating multiple sequence alignments)

FIG. 3. Scheme depicting a partial genomic DNA sequence of the bovine FcRn, which was PCR cloned applying the B7 (SEQ ID NO: 15) and B8 (SEQ ID NO: 16) primers. Capital letters indicate exons verified by cDNA sequence data. Exons and introns are numbered based on the genomic structure of the mouse FcRn (19). Diagonal breaks are added where segments of the sequence have been deleted for reasons of space. The dotted line indicates the splice acceptor site of intron 5, which carries the conserved AG dinucleotide but lacks the proper polypyrimidine tract, while the consensus splice acceptor site of intron 6 is highlighted by a dashed line. The splice acceptor site of intron 5 of mouse FcRn is in parenthesis under the bovine sequence indicating similarities between the two segments. Underlined letters in the mouse sequence indicate homology to the bovine splice acceptor site of intron 5 of the bovine gene.

FIG. 4. Tissue distribution of the two forms of bovine FcRn α-chain transcripts. A Northern blot analysis of a 1.6-kb transcript in 10 μg RNA from mammary gland (M), parotis (P), liver (L), jejunum (J), kidney (K), spleen (S) and from MDBK cell line (C) detected using a ³²P-labeled probe from the bFcRn transmembrane-cytoplasmic region. B RT-PCR analysis of the exon 6 deleted form of bFcRn transcript. Targeted PCR for exon 6 deleted cDNA amplification using B11/B12 primers (SEQ ID NO: 18 and 20, respectively).

FIG. 5. Functional expression of FcRn of different species in transfected cell lines. hFcRn/293 represents hFcRn transfected 293 cell line (7), 293 represents untransfected cells, B 1 represents bFcRn transfected rat IMCD cell lines, IMCD represents untransfected cells, rFcRn/IMCD represents rFcRn transfected IMCD cell line (14). Western blots of total cellular protein (10 μg per lane) by using affinity purified rabbit antisera raised against amino acids 173-187 (bovine residues) of the α2-α3 domains.

FIG. 6. Bovine-¹²⁵I-IgG binding by bFcRn transfected IMCD cell line. Assay were done at 37° C. with (filled columns) and without (open columns) competing unlabeled bovine IgG, at pH 6.0 or 8.0. Each column represents the mean cell-associated radioactivity in three replicates; bars show the standard error of the mean.

### DESCRIPTION OF EXPERIMENTS

Materials and Methods

Cloning of a bFcRn cDNA Fragment

RT-PCR—A bovine FcRn cDNA fragment was first cloned using reverse transcription-PCR (RT-PCR). Total RNA isolated from liver by TRIzol Reagent (Life Technologies, Inc., Gaithersburg, Md.) was reverse transcribed using a First-Strand cDNA Synthesis Kit (Pharmacia Biotech, Sweden). A segment spanning the α1, α2 and α3 domains was amplified by polymerase chain reaction using two degenerate primers (B3: 5′-CGCAGCARTAYCTGASCTACAA-3′ (SEQ ID NO: 7); B2: 5′-GATTCCSACCACRR-GCAC-3′(SEQ ID NO: 8)) which were designed based on the sequence homology of the published rat, mouse and human FcRn sequences (3, 5, 7).

Southern Blot Hybridization

The amplified cDNA was size fractionated on a 1-% agarose gel, blotted on a Hybond-N nylon membrane (Amersham, Arlington Heights, Ill.) and hybridized with a ³²P labeled human FcRn cDNA probe. This probe was generated by RT-PCR from placental RNA using primers (HUFC2: 5′-CCTGCTGGGCTGTGAACTG-3′(SEQ ID NO: 9); HUFC3: 5′-ACGGAGGACTTGGCTGGAG-3′(SEQ ID NO: 10)) and encompassed a 549 bp fragment containing the α2, α3 and transmembrane regions (7). Blots containing the fractionated PCR amplified product of bovine cDNA was hybridized in 5× Denhardt's solution, 5×SSC, 0.1% SDS, 100 μg/ml salmon sperm DNA at 60° C. for 6 hours and then washed in 2×SSC, 0.5% SDS for 2×15 min at room temperature, followed by a wash in 0.1×SSC, 0.1% SDS in 15 min at 60° C.

Cloning and Sequencing

Based on the expected size and Southern blot verification, the proper Taq polymerase generated fragment was cloned into the pGEM-T vector (Promega Corp., Madison, Wis.). In general, preliminary sequencing was done by fmol DNA Sequencing System (Promega Corp., Madison, Wash.) in the laboratory, while TaqFS dye terminator cycle sequencing was performed by an automated fluorescent sequencer (AB1, 373A-Stretch, Perkin Elmer) in the Cybergene company (Huddinge Sweden) to achieve the final sequence data

Cloning of the Full Length of bFcRn cDNA

To obtain the full length of bovine FcRn cDNA we used rapid amplification of the cDNA ends (RACE) technique (11) to isolate and clone the unknown 5′- and 3′-ends.

3′-RACE—5 μg of total RNA was reverse-transcribed by using Superscript II (Life Technologies, Inc., Gaithersburg, Md.) with the (dT) 17-adapter primer (5′-GACTCGAGTCGACATCGA(T)₁₇-3′(SEQ ID NO: 11)[used also for dromedary FcRn]). The resultant cDNA was then subjected to 3′RACE-PCR amplification using the adapter primer (5′-GACTCGAGTCGACATCG-3′(SEQ ID NO: 12) [used also for dromedary FcRn]) and a bFcRn specific primer (B3 (SEQ ID NO: 7)).

5′-RACE—The remaining 5′-end portion of the bovine FcRn was isolated using the 5′ RACE System for Rapid Amplification of cDNA Ends, Version 2.0 (Life Technologies, Inc., Gaithersburg, Md.). Briefly, total RNA was reverse transcribed using an FcRn-specific oligonucleotide (B4: 5′-GGCTCCTTCCACTCCAGGTT-3′(SEQ ID NO: 13)). After first strand synthesis, the original mRNA template was removed by treatment with the RNase mix. Unincorporated dNTPs, primer and proteins were separated from cDNA using a GlassMax Spin Cartridge. A homopolymeric tail was then added to the 3′-end of the cDNA using TdT and dCTP. PCR amplification was accomplished using Taq polymerase, a nested FcRn-specific primer (B5: 5′-CTGCTGCGTCCACTTGATA-3′(SEQ ID NO: 14)) and a deoxyinosine-containing anchor primer. The amplified cDNA segments were analyzed by Southern blot analysis, cloned and sequenced as described above.

Cloning of a bFcRn Genomic DNA Fragment

Bovine genomic DNA was purified from liver based on the method of Ausubel (12). To analyze exon-intron boundaries of the α3-transmembrane-cytoplasmic region we PCR amplified a genomic DNA fragment using the B7 (5′-GGCGACGAGCACCACTAC-3′(SEQ ID NO: 15)) and B8 (5′-GATTCCCGGAGGTCWCACA-3′(SEQ ID NO: 16)) primers. The amplified DNA was then ligated into the pGEM-T vector (Promega Corp., Madison, Wis.) and sequenced as described above.

Tissue Distribution

Northern Hybridization

Different bovine tissue samples (mammary gland, parotis, liver, jejunum, kidney and spleen) were collected at slaughter from a lactating Holstein-Fresian cow and frozen immediately in liquid nitrogen. Total cellular RNA purified from these tissues and from the MDBK cell line (TRIzol Reagent, Life Technologies, Inc., Gaithersburg, Md.) (10 μg/lane) was run on a denaturing agarose gel and transferred to a positively charged nylon membrane (Boehringer Mannheim GmbH, Germany). The blots were hybridized with a ³²P-labeled probe, which was generated by Prime-A-Gene kit (Promega Corp., Madison, Wis.), containing the B7-B8 (SEQ ID NO: 15-SEQ ID NO: 16) generated cDNA of the bFcRn. The final wash was 0.1×SSC, 0.1% SDS at 60° C.

Expression and Binding Assay

The full length of bFcRn cDNA was amplified by B10 (5′-CTGGGGCCGCAGA-GGGAAGG-3′(SEQ ID NO: 17) [used also for sheep FcRn gene]) and B11 (5′-GAGGCAGATCACAGGAGGAGAAAT-3′(SEQ ID NO: 18) [used also for sheep FcRn gene]). This segment was then cloned into the pCI-neo eucaryotic expression vector (Promega Corp., Madison, Wis.). 10 μg DNA was transfected into one 10 cm plate of IMCD cells using a CaPO₄ method (13). Cells were diluted and placed under G418 selection. Individual G418-resistant colonies were expanded for binding assays. The presence of the bovine FcRn in these cells was confirmed by Western blots.

Bovine IgG (Chemicon International, Temecula, Calif.) was labeled with Na¹²⁵I to a specific activity of ˜0.5 Ci/μmol using Iodogen (Pierce, Rockford, Ill.). pH dependent Fc binding and uptake was analyzed according to the protocol of Story et al. (7). Briefly, cells expressing the bovine FcRn were first washed with DMEM, pH 6 or 7.5. Then, bovine-¹²⁵I-IgG in DMEM, pH 6.0 or 7.5 with or without unlabeled bovine IgG was added. The cells were allowed to bind and take up IgG for 2 hours at 37° C. then unbound ligand was removed with washes of chilled PBS, pH 6.0 or 7.5. Bound radioligand was measured in a gamma counter.

Western Blot

A clone (B1) of IMCD cells transfected with cDNA encoding the bovine FcRn alpha chain, IMCD cells transfected with cDNA encoding the rat FcRn alpha chain (14), untransfected IMCD cells, 293 cells transfected with cDNA encoding the human FcRn alpha chain (7) and untransfected 293 cells were extracted in 5% SDS. Protein extracts were resolved on gradient polyacrylamide denaturing Tris-glycine gels (Novex, San Diego, Calif., USA) and transferred onto PVDF (Novex). Blots were probed with affinity-purified anti-FcRn peptide antibody, a rabbit antiserum against the peptide LEWKEPPSMRLKARP (SEQ ID NO: 19) representing amino acids 173-187 (bovine residues) of the α2-α3 domains (14) and bound antibody was detected with horse-radish peroxidase-conjugated goat anti-rabbit antibody and enhanced chemiluminescence (Renaissance Chemiluminescence Reagent; NEN Life Science Products Inc., Boston, Mass., USA).

Bio-Computing

Sequence comparison was completed by using the BLAST programs (15). Sequence pair distances—of bovine FcRn compare to other published FcRn sequences, was analysed by Megalign, Lasergene Biocomputing Software for the Macintosh (DNASTAR Inc., Madison, Wis.) using the J. Hein method (16) with PAM250 residue weight table.

Results

Isolation of the Bovine FcRn cDNA

To isolate a fragment of the bovine FcRn, we first synthesized cDNA from the RNA isolated from bovine liver, as this tissue was previously demonstrated to express FcRn in other species (6, 7). PCR amplification with two degenerate primers (B3 and B2; SEQ ID NO: 7 and 8, respectively) yielded a DNA fragment of about 750 bp. The degenerate primers were designed based on two conserved segments of rat (3), mouse (5) and human FcRn (7) sequences. Based on its expected size and the Southern blot verification with a cloned human FcRn fragment, this amplified DNA was ligated into a pGEM-T vector and one of the clones (clone 15/3) was completely sequenced. The data were compared to other GenBank sequences using the BLAST programs, and showed high homology to the human, rat and mouse FcRn cDNA. The insert of clone 15/3 started in the middle of the α1 domain (exon 3) and ended in the transmembrane region (exon 6).

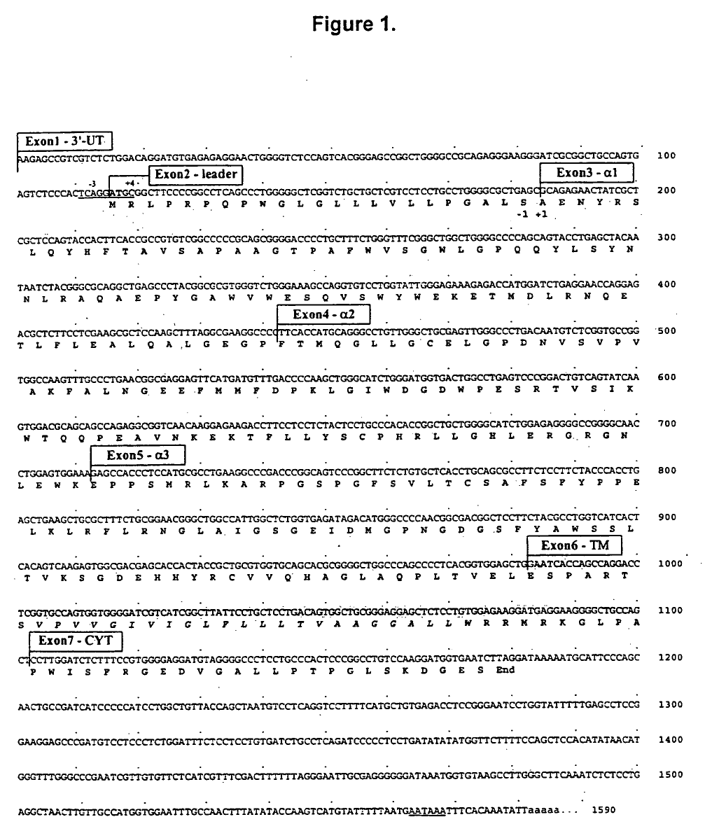

We then performed 3′-RACE, using B3 (SEQ ID NO: 7) and the adapter primer which generated a DNA fragment of ˜1.3 kbp. Several of the clones obtained were completely sequenced. One of these (clone 4), started in the middle of the α1 domain (exon 3) and ended with a 38-bp long poly(A) tail. The insert contained a segment of the α1, the full length of the α2, α3 domains, the transmembrane (TM) domain, the cytoplasmic (CYT) domain and ended with the 3′-untranslated (3′-UT) region. The total length of the insert was 1304 bp excluding the poly(A)-tail. Another clone (clone 1) revealed complete sequence homology to clone 4 but showed a 117 bp long deletion between the α3 domain and the cytoplasmic region. The total length of the insert was 1187 bp excluding the poly(A) tail. The 5′ portion of the bovine FcRn was obtained by applying a 5′-RACE technique. The amplification, in which we used B5 (SEQ ID NO: 14) and the adapter primers, produced a 600 bp DNA fragment, which then was ligated into the pGEM-T vector and one of the clones (clone 5) was sequenced. The insert of clone 5 contained 567 bp, which included the missing α1, signal, and 5′-untranslated (5′-UT) regions. Clones 5 and clone 4 had an overlap of 335 bp and therefore, it was possible to obtain a composite DNA sequence of 1582 bp, encompassing the entire region of the bovine FcRn cDNA³ (FIG. 1).

Characterization of Bovine FcRn cDNA

The sequenced and merged clones from 5′-RACE and 3′-RACE included a 116 bp long 5′-untranslated region followed by an ATG initiation codon. This motif is flanked by nucleotides which are important in the translational control in the Kozak consensus, CC^{A}/_{G}CCAUGG, the most important residues being the purine in position −3 and a G nucleotide in position +4 (17). The bovine FcRn cDNA shows TCAGGATGC which is different from the optimal Kozak sequence. Although, bFcRn shows a purine base in position −3 we found C instead of G in position +4 in all the clones we have sequenced from this animal (FIG. 1). To exclude the possibility of a Taq error during RT-PCR, we checked this motif from two other animals, and found the same sequence.

The initiation codon was followed by a 1180 bp or a 1063 bp long open reading frame in case of the full-length or the exon 6-deleted form, respectively. The exon-coded segment was followed by a 392-bp long 3′-untranslated sequence including a conserved polyadenylation signal.

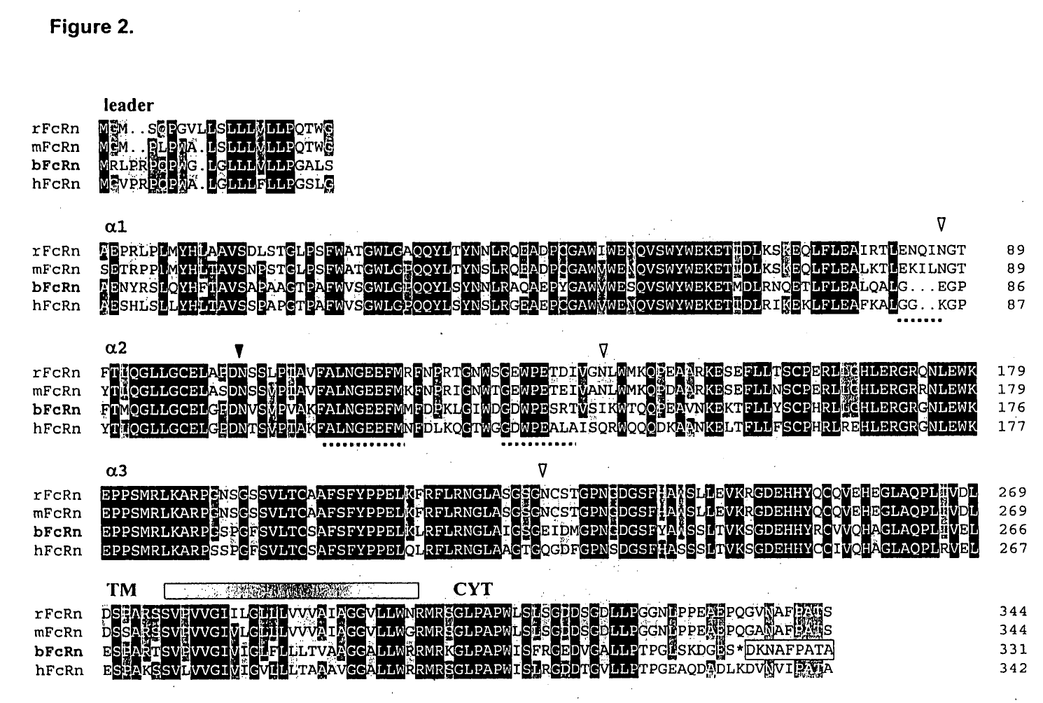

FIG. 2. shows the deduced amino acid sequence of the bovine FcRn (SEQ ID NO: 4) as compared to those of the human, rat and mouse. Previous studies indicate that the structure of the characterized FcRn molecules, resembles that of the MHC class-I α-chain (3, 18). The full length transcript of the bovine FcRn α-chain we isolated, is also composed of three extracellular domains (α1-α2-α3), a transmembrane region and a cytoplasmic tail. An exon 6-deleted transcript, though, lacks the putative transmembrane region. Except for this missing domain, the two molecules are identical at the DNA as well as at the protein level (FIG. 1).

Comparing the deduced bFcRn amino acid sequence (SEQ ID NO: 4) to its human, rat and mouse counterparts, we found the highest overall similarity to the human FcRn (Table 1). Among the extracellular domains, α3-chain turned to be the most conserved, while the cytoplasmic tail reflected the highest dissimilarity.

TABLE 1
Sequence pair distances (in percent similarity) of bovine
FcRn compared to published FcRn sequences, using the
J. Hein method with PAM250 residue weight table

| | α1 | α2 | α3 | TM | CYT | Total |
|---|---|---|---|---|---|---|
| Human | 75.6 | 74.4 | 85.6 | 74.4 | 61.5 | 77.1 |
| Mouse | 61.6 | 66.7 | 78.9 | 66.7 | 46.2 | 65.9 |
| Rat | 59.3 | 68.9 | 78.9 | 66.7 | 46.2 | 65.4 |

The high similarity of the bovine FcRn as compared to the human FcRn was further emphasized by analysing the end of the α1 domain. This segment, which forms a loop in the vicinity of the IgG binding site, shows a 3 or a 2 amino acid residue deletion, in the bovine and the human molecules respectively, compared to the rat and mouse sequences. Another common feature in these two molecules is that they show only one potential N-linked glycosylation site at amino acid residue 124, based on the bovine FcRn numbering system, compared to the rat or mouse counterparts where there are 3 additional sites (α1-domain: position 109; α2-domain: position 150; α3-domain: position 247 based on the rat FcRn numbering system).

In contrast to the known FcRn sequences, we found an unusually short cytoplasmic tail in the bFcRn where this segment is composed of 30 rather than 40 amino acid residues as in all other FcRn molecules so far analyzed. Despite its shortness, the cytoplasmic tail of the bFcRn shows the di-leucine motif (aa: 319-320) which was previously identified as a critical signal for endocytosis but not for basolateral sorting, although, similar to the human molecule, it lacks the casein kinase II (CKII) phosphorylation site, which is found in the rat FcRn upstream of the di-leucine motif.

Interestingly, the nucleotides which follow the stop signal represent codons for similar amino acid residues which are found at the 3′ end of the human, rat and mouse molecules (FIG. 2, residues in rectangle in the bovine sequence), although it lacks the stop signal at the end of this segment which is shared in the other FcRns. Finding this sequence in all the clones we have analyzed and the lack of the common stop signal in the expected conserved position, exclude the possibility of a Taq error due to the 3′-RACE (RT-PCR) process and suggests that a mutation has occurred in this part of the gene.

Genomic DNA Segment of bFcRn

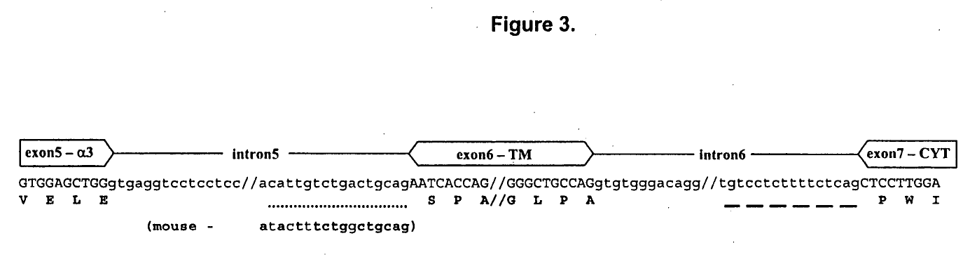

The two different transcripts of the bFcRn were compared to the published mouse genomic sequence (19). Analysis of the mouse exon-intron boundaries around α3-TM-CYT domains suggested that exon 6 is completely eliminated from the bovine transcript representing clone 1. To verify this hypothesis, we cloned the genomic segment of the region of interest which contained part of exon 5, exon 6 and a short fragment of exon 7 and the two introns (intron 5 and intron 6). The B7/B8 (SEQ ID NO: 15/16) amplified DNA was then cloned and sequenced. The nucleotide sequences surrounding the exon-intron boundaries revealed that the bovine splicing sites agree with their mouse counterparts (FIG. 3). Analyzing the 5′ splice site (donor site) and the 3′ splice site (acceptor site) of intron 5 and intron 6, we found that intron 5 has a conserved splice donor site (GT) while its 3′ splice site differs from the consensus splice acceptor sequence, which is composed of a polypyrimidine tract (PPyT) followed by an AG dinucleotide. Although the acceptor site of intron 5 has the conserved AG dinucleotide it lacks the conserved polypyrimidine tract. This non-conserved splice acceptor site of intron 5 shows similarity to the same gene segment of the mouse FcRn since it shows only 4 differences from the 1.5 nucleotides preceding the AG dinucleotide motif (FIG. 3). Despite this similarity, though, there is a 14 nt long conserved PPyT in the mouse intron, followed by 24 nt and then the AG dinucleotide (19). A similar sequence was not detected at the 3′ end of the bovine intron 5 (5′ . . . ctgtctggat ctctggtgga ggactcgacc ccatccctgt cctgactcag atctgcgagg cccttaaata tctcacaaca ttgtctgact gcagAATCACCAGCC . . . ), whereas the splice donor and splice acceptor sites of intron 6 shows conserved boundary sequences.

Tissue Distribution of the Two Forms of Bovine FcRn α-Chain Transcript

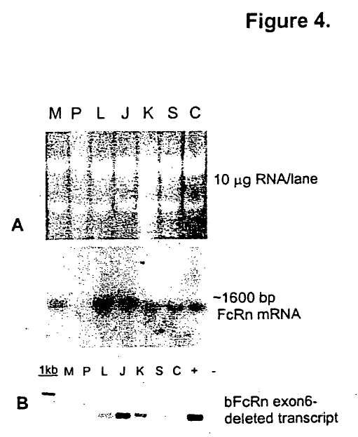

We then examined the tissue distribution of the two forms of the bFcRn α chain mRNA by using Northern blots and RT-PCR Based on the Northern blot analyses, a 1.6-kb transcript was present in RNA from mammary gland, liver, jejunum, kidney, and spleen from a normal lactating Holstein Friesian cow and the MDBK cell line (FIG. 4.A) at different levels of expression, whereas we did not find expression in parotis. The signal could not represent cross-hybridization with class-I MHC mRNA since it was detected with a probe from the transmembrane-cytoplasmic-3′-UT region, which is dissimilar from the class I sequences. Although, this probe is able to detect both forms of the bFcRn, we were unable to detect the shorter transmembrane-exon-deleted form, probably because of its low expression level or due to the low resolution of the gel electrophoresis.

In order to analyze the expression of the alternatively spliced—exon 6-deleted—transcript in tissues listed above, we performed a targeted PCR amplification (20) in which we used primer B11 (SEQ ID NO: 18) and B12 (SEQ ID NO:20). B12 corresponds to the 5′ boundary conserved region of exon 5 juxtaposed with two conserved nucleotides in 3′ boundary region of exon 7. This amplification detected exon 6-deleted transcripts in all tissues tested (FIG. 4.B).

Expression and IgG Binding of Bovine FcRn α Chain in Transfected Cells

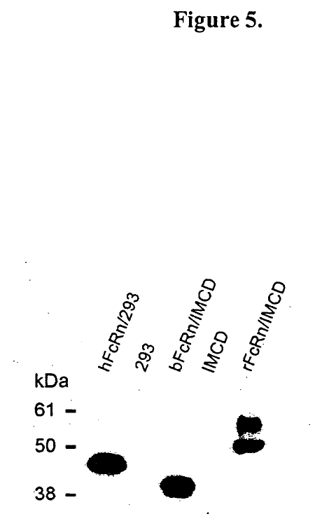

FcRn tranfected cell lines were assessed by Western blot using rabbit antipeptide antisera raised against an epitope of human FcRn heavy chain (amino acids 174-188). Since this epitope is common in the human, in the rat and in the bovine FcRn molecules, we used this antibody to detect the expressed bovine FcRn, as well as its human and rat counterparts, as controls. We detected a ˜45 kDa band in the hFcRn transfected human embryonic kidney 293 cell line, a ˜40 kDa band in the bFcRn transfected IMCD cell lines, and two bands (˜50 kDa, and ˜55 kDa) in the rFcRn transfected IMCD cell line. The 45 kDa and the 50 kDa, 55 kDa bands detected of the human and rat FcRn transfected cells, are consistent with the known molecular weight of the human and the rat FcRn α chains (6, 21), respectively. The lower band in the rat FcRn transfected IMCD cell line is the high mannose form of FcRn usually found in endoplasmic reticulum, whereas FcRn in the upper band contains complex-type oligosaccharide chains modified in the Golgi. There was no hybridization in the untransfected 293 and IMCD cells (FIG. 5).

The nearly 40 kDa band we detected in the bovine FcRn transfected IMCD cell line indicates that the cDNA we isolated as bovine FcRn is intact and can be fully translated. The lower moleculer weight of the bovine FcRn compare to the human and rat molecules is probably due to its shorter cytoplasmic region and/or different glycosylation.

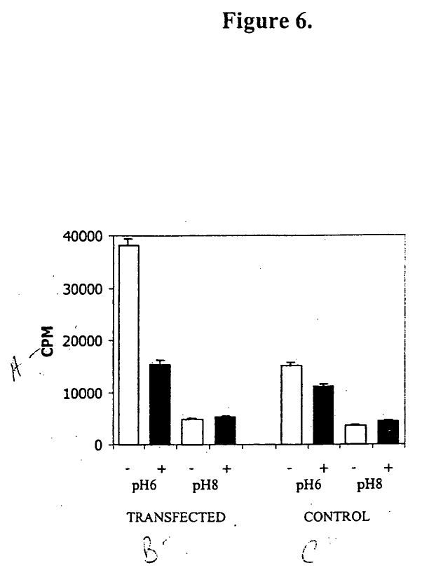

To determine whether the bovine FcRn clone encoded an Fc receptor, we measured the binding of radiolabeled bovine IgG on the bFcRn transfected rat IMCD cell line (B1). Cells that expressed bFcRn bound IgG specifically at pH6.0 but not at pH7.5; untransfected cells showed little or no specific binding at either pH (FIG. 6). A similar pH dependence of binding has previously been observed for human (7) and rat FcRn (22).

Summary of Results

The predominance of IgG1 in lacteal fluid, intestinal secretions, respiratory fluid and lacrimal fluid supports the concept of a special role for IgG 1 in mucosal immunity in cattle. The higher ratio of IgG1:IgG2 in these secretions when compared to serum could represent a combination of selective IgG1 transport and local synthesis. Immunoglobulin transmission through the mammary epithelial cells is by far the most studied, since in the cow, maternal immunity is exclusively mediated by colostral immunoglobulins. The receptor responsible for the IgG transport has not been identified prior to the present invention, although previous studies have indicated that specific binding sites exist on bovine mammary epithelial cells near parturition which are presumably involved in the transfer of IgG1. We have now isolated and characterized a cDNA encoding a bovine homologue of the human, rat and mouse IgG transporting Fc receptor, FcRn.

Sequence Analysis

Extracellular Backbone and the FcRn/Fc Interaction Site

The bovine cDNA and its deduced amino acid sequence were similar to the rat, mouse and human FcRns (FIG. 2) (3, 5, 7). Among these sequences, the bovine α chain shows the highest overall similarity to its human counterpart (Table 1).

Based on the crystal structure of a 2:1 complex of FcRn and the Fc fragment of rat IgG (18) the approximate binding region on each molecule could be localized. The first contact zone of the heavy chain of the rat FcRn molecule can be found at the end of the α1 domain involving residues 84-86, and 90. The second contact zone involves residues 113-119, while the third contact zone encompasses residues 131-137, both segments are part of the α2 domain.

The close relationship between the human and bovine FcRn molecules was further emphasized by analyzing the end of the α1 domain, which was suspected to form the first contact zone in the rat FcRn/Fc interaction. Both the bovine and human FcRns are three and two amino acid residues shorter, respectively, compared to their rodent counterparts. It is interesting that these deletions eliminate an N-linked glycosylation site found in their rat and mouse counterparts and which is ubiquitous in MHC class-I proteins.

The second contact zone, which is part of the α2 domain, is well conserved, emphasizing its importance in IgG binding. Another difference of the bovine FcRn compared to the rat molecule is a radical amino acid substitution at the third contact zone (aa: 134-Arg) in the α2 domain. These observations suggest critical importance of the second and third contact zones, while those residues that make up the first contact zone are probably less crucial in the IgG/FcRn interaction in the cow and also in humans, further supporting the conclusion of Vaughn et al. (24) who applied site directed mutagenesis to analyze the role of the predicted contact residues of the rat FcRn. They found that replacement of residues 84-86 of the α1 domain, which was thought to be the first contact zone, did not significantly alter binding affinity.

We found that the critical residues of the α3 domains (aa: 216L, 242K, 248H, 249H), which also influence the FcRn/Fc interaction are conserved among the different species thus far analyzed. The bFcRn, similarly to its human counterpart, has an absence of the N-linked glycosylation site in the α3 domain, which is of interest, since for rat FcRn this has been suggested to mediate FcRn dimerization via a carbohydrate handshake (22).

In this context one might predict that in the cow, the mammary epithelial cells are able to carry IgG via FcRn mediated transcytosis from the blood into their secretory fluid, although none of the studies indicated pH dependent IgG binding, which we found in analyzing IgG binding to the bovine FcRn (FIG. 6).

In summary, our data indicate that the FcRn transcripts are expressed in different tissues, including the mammary gland, in cattle, and strengthens their suggested involvement in IgG catabolism and transcytosis (for review see Junghans, 1997 (23)). It will be of interest to investigate the bFcRn binding affinity or the transport efficiency mediated by this receptor of the bovine IgG subclasses. Analyses of the localization and the expressional level of the bFcRn in the mammary gland at different times during the lactation period may also help to clarify its function in the transport of IgG into the colostrum.

Production of Proteins Fused to Immunoglobulin γ-Chains

Examples of techniques of producing proteins fused to immunoglobulin γ-chains are described in a number of publications (e.g. 24-35) and will therefore not be described herein.

Production of Transgenic Ruminants

Examples of techniques of producing transgenic animals are disclosed in many prior art publications (e.g. transgenic sheep (36-52) and transgenic cows (53-67)) and will not be described herein.

However, the teachings of all references cited in the present specification are hereby included by reference.

### REFERENCES

1. Brambell, F. W. 1969. The transmission of immune globulins from the mother to the foetal and newborn young. Proc Nutr Soc 28:35.
2. Larson, B. L., H. L. Heary, Jr., and J. E. Devery. 1980. Immunoglobulin production and transport by the mammary gland. J Dairy Sci 63:665.
3. Simister, N. E., and K. E. Mostov. 1989. An Fc receptor structurally related to MHC class I antigens. Nature 337:184.
4. Rodewald, R., and J. P. Kraehenbuhl. 1984. Receptor-mediated transport of IgG. J Cell Biol 99:159s.
5. Ahouse, J. J., C. L. Hagerman, P. Mittal, D. J. Gilbert, N. G. Copeland, N. A. Jenkins, and N. E. Simister. 1993. Mouse MHC class I-like Fc receptor encoded outside the MHC. J Immunol 151:6076.
6. Blumberg, R. S., T. Koss, C. M. Story, D. Barisani, J. Polischuk, A. Lipin, L. Pablo, R. Green, and N. E. Simister. 1995. A major histocompatibility complex class I-related Fc receptor for IgG on rat hepatocytes. J Clin Invest 95:2397.
7. Story, C. M., J. E. Mikulska, and N. E. Simister. 1994. A major histocompatibility complex class I-like Fc receptor cloned from human placenta: possible role in transfer of immunoglobulin G from mother to fetus. J Exp Med 180:2377.
8. Simister, N. E., C. M. Story, H. L. Chen, and J. S. Hunt. 1996. An IgG-transporting Fc receptor expressed in the syncytiotrophoblast of human placenta. Eur J Immunol 26:1527.
9. Cianga, P., C. Medesan, J. A. Richardson, V. Ghetie, and E. S. Ward. 1999. Identification and function of neonatal Fc receptor in mammary gland of lactating mice. Eur J Immunol 29:2515.
10. Medesan, C., P. Cianga, M. Mummert, D. Stanescu, V. Ghetie, and E. S. Ward. 1998. Comparative studies of rat IgG to further delineate the Fc:FcRn interaction site. Eur J Immunol 28:2092.
11. Frohman, M. A., M. K. Dush, and G. R. Martin. 1988. Rapid production of full-length cDNAs from rare transcripts: amplification using a single gene-specific oligonucleotide primer. Proc Natl Acad Sci USA 85:8998.
12. Ausubel, F. M., R. Brent, R. E. Kingston, D. D. Moore, J. G. Seidman, J. A. Smith, and K. Struhl. 1989. Current protocols in Molecular Biology. Greene Publishing Associates and Wiley-Interscience, John Wiley & Sons.
13. Gorman, C. M., D. R. Gies, and G. McCray. 1990. Transient production of proteins using an adenovirus transformed cell line. DNA Prot Eng Tech 2:3.
14. McCarthy, K. M., Y. Yoong, and N. E. Simister. 1999. Bidirectional trancytosis of IgG by the rat neonatal. Fc receptor expressed in a rat kidney cell line: a system to study protein transport across epithelia. J Cell Sci 113:1277-1285.
15. Altschul, S. F., W. Gish, W. Miller, E. W. Myers, and D. J. Lipman. 1990. Basic local alignment search tool. J Mol Biol 215:403.
16. Hein, J. 1990. Unified approach to alignment and phylogenies. In Methods in Enzimology, vol. 183. Academic Press, Inc., San Diego, Calif., p. 626.
17. Kozak, M. 1986. Point mutations define a sequence flanking the AUG initiator codon that modulates translation by eukaryotic ribosomes. Cell 44:283.
18. Burmeister, W. P., A. H. Huber, and P. J. Bjorkman. 1994. Crystal structure of the complex of rat neonatal Fc receptor with Fc. Nature 372:379.
19. Kandil, E., M. Noguchi, T. Ishibashi, and M. Kasahara. 1995. Structural and phylogenetic analysis of the MHC class I-like Fc receptor gene. J Immunol 154:5907.
20. Yang, D., and J. Le. 1994. Targeted amplification of alternatively spliced transcripts of major histocompatibility complex class I heavy chain. J Immunol Methods 176:265.
21. Dickinson, B. L., K. Badizadegan, Z. Wu, J. C. Ahouse, X. Zhu, N. E. Simister, R. S. Blumberg, and W. I. Lencer. 1999. Bidirectional FcRn-dependent IgG transport in a polarized human intestinal epithelial cell line. J Clin Invest 104:903.
22. Vaughn, D. E., and P. J. Bjorkman. 1998. Structural basis of pH-dependent antibody binding by the neonatal Fc receptor. Structure 6:63.
23. Junghans, R. P. 1997. Finally! The Brambell Receptor (FcRB). Immunol Res 16:29.
24. Lawson B R., G J. Prud'homme, Y. Chang, H A. Gardner, J. Kuan, D H. Kono, and A N. Theofilopoulos. 2000. Treatment of murine lupus with cDNA encoding IFN-gammaR/Fc [see comments]. Journal of Clinical Investigation. 106(2):207-15.
25. Winter C C., and E O. Long. 2000. Binding of soluble KIR-Fc fusion proteins to HLA class I. [Review] [16 refs] Methods in Molecular Biology. 121:239-50.
26. Feng X., X X. Zheng, S. Yi, A M. Lehnert, T B. Strom, and P J. O'Connell. 1999. IL-10/Fc inhibits macrophage function and prolongs pancreatic islet xenograft survival. Transplantation. 68(11):1775-83.
27. Zheng X X., A W. Steele, W W. Hancock, K. Kawamoto, X C. Li, P W. Nickerson, Y. Li, Y. Tian, and TB. Strom. 1999. IL-2 receptor-targeted cytolytic IL-2/Fc fusion protein treatment blocks diabetogenic autoimmunity in nonobese diabetic mice. Journal of Immunology. 163(7):4041-8.
28. Feng S., R R. Quickel, J. Hollister-Lock, M. McLeod, S. Bonner-Weir, R C. Mulligan, and G C. Weir. 1999. Prolonged xenograft survival of islets infected with small doses of adenovirus expressing CTLA4Ig. Transplantation. 67(12):1607-13.
29. Knechtle S J., A D. Kirk, J H Jr. Fechner, X. Hong, Y. Dong, M M. Hamawy, and D M. Harlan. 1999. Inducing unresponsiveness by the use of anti-CD3 immunotoxin, CTLA4-Ig, and anti-CD40 ligand. Transplantation Proceedings. 31(3B Suppl):27S-28S.
30. Novak E J., C M. Blosch, J D. Perkins, C L. Davis, D. Barr, J P. McVicar, R S. Griffin, A L. Farrand, M. Wener, and C L. Marsh. 1998. Recombinant human tumor necrosis factor receptor Fc fusion protein therapy in kidney transplant recipients undergoing OKT3 induction therapy. Transplantation. 66(12):1732-5.
31. Lo K M., Y. Su-do, J. Chen, Y. Li, Y. Lan, S M. Kong, L. Chen, Q. An, and S D. Gillies, 1998. High level expression and secretion of Fc-X fusion proteins in mammalian cells. Protein Engineering. 11(6):495-500.
32. Komesli S., D. Vivien, and P. Dutartre 1998. Chimeric extracellular domain type II transforming growth factor (TGF)-beta receptor fused to the Fc region of human immunoglobulin as a TGF-beta antagonist. European Journal of Biochemistry. 254(3):505-13.
33. Tran H M., P W. Nickerson, A C. Restifo, M A. Ivis-Woodward, A. Patel, R D. Allen, T B. Strom, and O'Connell P J. 1997. Distinct mechanisms for the induction and maintenance of allograft tolerance with CTLA4-Fc treatment. Journal of Immunology. 159(5):2232-9.
34. Moreland L W., S W. Baumgartner, M H. Schiff, E A. Tindall, R M. Fleischmann, A L. Weaver, R E. Ettlinger, S. Cohen, W J. Koopman, K. Mohler, M B. Widmer, and C M. Blosch. 1997. Treatment of rheumatoid arthritis with a recombinant human tumor necrosis factor receptor (p75)-Fc fusion protein [see comments]. New England Journal of Medicine. 337(3):141-7.
35. Fidel P L Jr., R. Romero, J. Cutright, N. Wolf, R. Gomez, H. Araneda, M. Ramirez, and B H. Yoon. 1997. Treatment with the interleukin-1 receptor antagonist and soluble tumor necrosis factor receptor Fc fusion protein does not prevent endotoxin-induced preterm parturition in mice. Journal of the Society for Gynecologic Investigation. 4(1):22-6.
36. Ward K A., and B W. Brown. 1998. The production of transgenic domestic livestock: successes, failures and the need for nuclear transfer. [Review] [24 refs] Reproduction, Fertility, & Development. 10(7-8):659-65.
37. Pintado B., and A. Gutierrez-Adan. 1999. Transgenesis in large domestic species: future development for milk modification. [Review] [65 refs] Reproduction, Nutrition, Development. 39(5-6): 535-44.
38. Niemann H., R. Halter, J W. Carnwath, D. Herrmann, E. Lemme, and D. Paul. 1999. Expression of human blood clotting factor VIII in the mammary gland of transgenic sheep. Transgenic Research. 8(3):23747.
39. Dalrymple M A., and I. Garner. 1998. Genetically modified livestock for the production of human proteins in milk. [Review] [63 refs] Biotechnology & Genetic Engineering Reviews. 15:33-49.
40. Clark A J. 1998. Gene expression in the mammary glands of transgenic animals. [Review] [29 refs] Biochemical Society Symposia. 63:133-40.
41. Schnieke A E., A J. Kind, W A. Ritchie, K. Mycock, A R. Scott, M. Ritchie, I. Wilmut, A. Colman, and K H. Campbell. 1997. Human factor IX transgenic sheep produced by transfer of nuclei from transfected fetal fibroblasts [see comments]. Science. 278(5346):2130-3.
42. Butler S P., K. van Cott, A. Subrumanian, F C. Gwazduaskas, and W H. Velander. 1997. Current progress in the production of recombinant human fibrinogen in the milk of transgenic animals. [Review] [25 refs] Thrombosis & Haemostasis. 78(1):537-42.
43. Maga E A., and J D. Murray. 1995. Mammary gland expression of transgenes and the potential for altering the properties of milk. [Review] [110 refs] Bio/Technology. 13(13):1452-7.
44. Houdebine L M. 1995. The production of pharmaceutical proteins from the milk of transgenic animals. [Review] [64 refs] Reproduction, Nutrition, Development. 35(6):609-17.
45. Janne J., J M. Hyttinen, T. Peura, M. Tolvanen, L. Alhonen, R. Sinervirta, and M. Halmekyto. 1994. Transgenic bioreactors. [Review] [54 refs] International Journal of Biochemistry. 26(7):859-70.
46. Houdebine L M. 1994. Production of pharmaceutical-proteins from transgenic animals. [Review] [133 refs] Journal of Biotechnology. 34(3):269-87.
47. Carver A S., M A. Dalrymple, G. Wright, D S. Cottom, D B. Reeves, Y H. Gibson, J L. Keenan, J D. Barrass, A R. Scott, and A. Colman, et al. 1993. Transgenic livestock as bioreactors: stable expression of human alpha-1-antitrypsin by a flock of sheep. Bio/Technology. 11(11):1263-70.
48. Yom H C., and R D. Bremel. 1993. Genetic engineering of milk composition: modification of milk components in lactating transgenic animals. [Review] [54 refs] American Journal of Clinical Nutrition. 58(2 Suppl):299S-306S.
49. Wall R J., H W. Hawk, and N. Nel. 1992. Making transgenic livestock: genetic engineering on a large scale [see comments]. [Review] [45 refs] Journal of Cellular Biochemistry. 49(2):113-20.
50. Wright G., A. Carver, D. Cottom, D. Reeves, A. Scott, P. Simons, I. Wilmut, I. Garner, and Colman A. 1991. High level expression of active human alpha-1-antitrypsin in the milk of transgenic sheep. Bio/Technology. 9(9):830-4.
51. Rexroad C E Jr. 1991. Production of sheep transgenic for growth hormone genes. [Review] [16 refs] Biotechnology. 16:259-63.
52. Murray J D., C D. Nancarrow, J T. Marshall, I G. Hazelton, and K A. Ward. 1989. Production of transgenic merino sheep by microinjection of ovine metallothionein-ovine growth hormone fusion genes. Reproduction, Fertility, & Development. 1(2):147-55.
53. Shemesh M., M. Gurevich, E. Harel-Markowitz, L. Benvenisti, L S. Shore, and Y. Stram. 2000. Gene integration into bovine sperm genome and its expression in transgenic offspring. Molecular Reproduction & Development. 56(2 Suppl):306-8.
54. Brink M F., M D. Bishop, and F R. Pieper. 2000. Developing efficient strategies for the generation of transgenic cattle which produce biopharmaceuticals in milk. [Review] [29 refs] Theriogenology. 53(1):139-48.
55. Eyestone W H. 1999. Production and breeding of transgenic cattle using in vitro embryo production technology. Theriogenology. 51(2):509-17.
56. Zuelke K A. 1998. Transgenic modification of cows milk for value-added processing. [Review] [62 refs] Reproduction, Fertility, & Development. 10(7-8):671-6.
57. Pintado B., and A. Gutierrez-Adan. 1999. Transgenesis in large domestic species: future development for milk modification. [Review] [65 refs] Reproduction, Nutrition, Development. 39(5-6): 535-44.
58. Chan A W., E J. Homan, L U. Ballou, J C. Burns, and RD. Bremel. 1998. Transgenic cattle produced by reverse-transcribed gene transfer in oocytes. Proceedings of the National Academy of Sciences of the United States of America. 95(24):14028-33.
59. Dalrymple M A., and I. Garner. 1998. Genetically modified livestock for the production of human proteins in milk. [Review] [63 refs] Biotechnology & Genetic Engineering Reviews. 15:33-49.
60. Wall R J., D E. Kerr, and K R. Bondioli. 1997. Transgenic dairy cattle: genetic engineering on a large scale. [Review] [85 refs] Journal of Dairy Science. 80(9):2213-24.
61. Haskell R E., and R A. Bowen. 1995. Efficient production of transgenic cattle by retroviral infection of early embryos. Molecular Reproduction & Development. 40(3):386-90.
62. Hyttinen J M., T. Peura, M. Tolvanen, J. Aalto, L. Alhonen, R. Sinervirta, M. Halmekyto, S. Myohanen, and J. Janne. 1994. Generation of transgenic dairy cattle from transgene-analyzed and sexed embryos produced in vitro. Bio/Technology. 12(6):606-8.
63. Houdebine L M. 1994. Production of pharmaceutical proteins from transgenic animals. [Review] [133 refs] Journal of Biotechnology. 34(3):269-87.
64. Bowen R A., M L. Reed, A. Schnieke, G E Jr. Seidel, A. Stacey, W K. Thomas, and O. Kajikawa. 1994. Transgenic cattle resulting from biopsied embryos: expression of c-ski in a transgenic calf. Biology of Reproduction. 50(3):664-8.
65. Krimpenfort P. 1993. The production of human lactoferrin in the milk of transgenic animals. Cancer Detection & Prevention. 17(2):301-5.
66. Krimpenfort P., A. Rademakers, W. Eyestone, A. van der Schans, S. van den Broek, P. Kooiman, E. Kootwijk, G. Platenburg, F. Pieper, and R. Strijker, et al. 1991. Generation of transgenic dairy cattle using ‘in vitro’ embryo production. Bio/Technology. 9(9):844-7
67. Bondioli K R, K A. Biery, K G. Hill, and K B. Jones. 1991. De Mayo F J. Production of transgenic cattle by pronuclear injection. Biotechnology. 16:265-73.

1. A method of producing colostrums or milk with enhanced levels of immunoglobulins or proteins fused to immunoglobulin γ-chains or FcRn interacting parts thereof, comprising the steps of transferring an immunoglobulin G (IgG) transporting ruminant Fc receptor (FcRn) α-chain DNA molecule through transient or persistent transgenesis into the corresponding ruminant animal for overexpression of the protein expressed by the ruminant FcRn α-chain DNA molecule, optionally at concomitant upregulation of the expression of the corresponding β2-microglobulin gene, to increase the number of functional receptors in the udder, thereby enhancing the transport of immunoglobulins and/or proteins fused to immunoglobulin γ-chains or FcRn interacting parts thereof from, or through, the udder into the colostrums or milk.

2. The method according to claim 1, wherein the ruminant of the immunoglobulin G (IgG) transporting ruminant Fc receptor (FcRn) α-chain DNA molecule is selected from the group consisting of cow, dromedary and sheep.

3. The method according to claim 2, wherein the DNA molecule has a nucleotide sequence selected from the group consisting of SEQ ID NO: 1, SEQ ID NO: 2, SEQ ID NO: 3, and modified sequences of these three sequences expressing proteins with IgG transporting function.

4. The method according to claim 1, wherein the ruminant of the protein expressed by the ruminant FcRn α-chain DNA molecule is selected from the group consisting of cow, dromedary and sheep.

5. The method according to claim 4, wherein the protein has an amino acid sequence selected from the group consisting of SEQ ID NO: 4, SEQ ID NO: 5, SEQ ID NO: 6, and modified sequences of these three sequences with IgG transporting function.


SEQUENCE LISTING (137 sequences)

SEQ ID NO: 1 - DNA (genomic), 14 bp; unknown-stranded, unknown
  hypothetical: YES
tgcaggtggatagt

SEQ ID NO: 2 - DNA (genomic), 18 bp; unknown-stranded, unknown
  hypothetical: YES
catgtcgatagtcttgca

SEQ ID NO: 3 - DNA (genomic), 14 bp; unknown-stranded, unknown
  hypothetical: YES
gtcgatagtcttgc

SEQ ID NO: 4 - DNA (genomic), 14 bp; unknown-stranded, unknown
  hypothetical: YES
ccatgtcgatagtc

SEQ ID NO: 5 - DNA (genomic), 14 bp; unknown-stranded, unknown
  hypothetical: YES
ctccatgtcgatag

SEQ ID NO: 6 - DNA (genomic), 14 bp; unknown-stranded, unknown
  hypothetical: YES
cttggacaggatct

SEQ ID NO: 7 - DNA (genomic), 14 bp; unknown-stranded, unknown
  hypothetical: YES
tgctgttgtacagg

SEQ ID NO: 8 - DNA (genomic), 14 bp; unknown-stranded, unknown
  hypothetical: YES
gtgctgttgtacag

SEQ ID NO: 9 - DNA (genomic), 14 bp; unknown-stranded, unknown
  hypothetical: YES
ttggcgtagtagtc

SEQ ID NO: 10 - DNA (genomic), 14 bp; unknown-stranded, unknown
  hypothetical: YES
tccaccattagcac

SEQ ID NO: 11 - DNA (genomic), 14 bp; unknown-stranded, unknown
  hypothetical: YES
gatttcgttgtggg

SEQ ID NO: 12 - DNA (genomic), 18 bp; unknown-stranded, unknown
  hypothetical: YES
gtcatagatttcgttgtg

SEQ ID NO: 13 - DNA (genomic), 16 bp; unknown-stranded, unknown
  hypothetical: YES
tgtactctgcttgaac

SEQ ID NO: 14 - DNA (genomic), 14 bp; unknown-stranded, unknown
  hypothetical: YES
gtgtactctgcttg

SEQ ID NO: 15 - DNA (genomic), 14 bp; unknown-stranded, unknown
  hypothetical: YES
tgctgtgtgtactc

SEQ ID NO: 16 - DNA (genomic), 18 bp; unknown-stranded, unknown
  hypothetical: YES
ctgatgtgttgaagaaca

SEQ ID NO: 17 - DNA (genomic), 16 bp; unknown-stranded, unknown
  hypothetical: YES
ctctgatgtgttgaag

SEQ ID NO: 18 - DNA (genomic), 14 bp; unknown-stranded, unknown
  hypothetical: YES
gctctgatgtgttg

SEQ ID NO: 19 - DNA (genomic), 14 bp; unknown-stranded, unknown
  hypothetical: YES
gagctctgatgtgt

SEQ ID NO: 20 - DNA (genomic), 18 bp; unknown-stranded, unknown
  hypothetical: YES
cacttttaacttgagcct

SEQ ID NO: 21 - DNA (genomic), 18 bp; unknown-stranded, unknown
  hypothetical: YES
ctccacttttaacttgag

SEQ ID NO: 22 - DNA (genomic), 18 bp; unknown-stranded, unknown
  hypothetical: YES
tgctgtatttctggtaca

SEQ ID NO: 23 - DNA (genomic), 14 bp; unknown-stranded, unknown
  hypothetical: YES
ccaggaattgttgc

SEQ ID NO: 24 - DNA (genomic), 14 bp; unknown-stranded, unknown
  hypothetical: YES
ttgctgaggtatcg

SEQ ID NO: 25 - DNA (genomic), 14 bp; unknown-stranded, unknown
  hypothetical: YES
gataaccactctgg

SEQ ID NO: 26 - DNA (genomic), 18 bp; unknown-stranded, unknown
  hypothetical: YES
caaaagataaccactctg

SEQ ID NO: 27 - DNA (genomic), 15 bp; unknown-stranded, unknown
  hypothetical: YES
cggtgacatcaaaag

SEQ ID NO: 28 - DNA (genomic), 14 bp; unknown-stranded, unknown
  hypothetical: YES
cctcaatttcccct

SEQ ID NO: 29 - DNA (genomic), 14 bp; unknown-stranded, unknown
  hypothetical: YES
gttatccctgctgt

SEQ ID NO: 30 - DNA (genomic), 14 bp; unknown-stranded, unknown
  hypothetical: YES
gcagtgtgttatcc

SEQ ID NO: 31 - DNA (genomic), 14 bp; unknown-stranded, unknown
  hypothetical: YES
gatgtccacttgca

SEQ ID NO: 32 - DNA (genomic), 14 bp; unknown-stranded, unknown
  hypothetical: YES
tagtgaacccgttg

SEQ ID NO: 33 - DNA (genomic), 14 bp; unknown-stranded, unknown
  hypothetical: YES
tgccatgaatggtg

SEQ ID NO: 34 - DNA (genomic), 16 bp; unknown-stranded, unknown
  hypothetical: YES
gttcatgccatgaatg

SEQ ID NO: 35 - DNA (genomic), 14 bp; unknown-stranded, unknown
  hypothetical: YES
catgagaagcagga

SEQ ID NO: 36 - DNA (genomic), 14 bp; unknown-stranded, unknown
  hypothetical: YES
gctttgcagatgct

SEQ ID NO: 37 - DNA (genomic), 14 bp; unknown-stranded, unknown
  hypothetical: YES
gagctttgcagatg

SEQ ID NO: 38 - DNA (genomic), 14 bp; unknown-stranded, unknown
  hypothetical: YES
tagttggtgtccag

SEQ ID NO: 39 - DNA (genomic), 16 bp; unknown-stranded, unknown
  hypothetical: YES
ctgaagcaatagttgg

SEQ ID NO: 40 - DNA (genomic), 18 bp; unknown-stranded, unknown
  hypothetical: YES
agctgaagcaatagttgg

SEQ ID NO: 41 - DNA (genomic), 14 bp; unknown-stranded, unknown
  hypothetical: YES
ggagctgaagcaat

SEQ ID NO: 42 - DNA (genomic), 14 bp; unknown-stranded, unknown
  hypothetical: YES
caatgtacagctgc

SEQ ID NO: 43 - DNA (genomic), 16 bp; unknown-stranded, unknown
  hypothetical: YES
ggaagtcaatgtacag

SEQ ID NO: 44 - DNA (genomic), 15 bp; unknown-stranded, unknown
  hypothetical: YES
cggaagtcaatgtac

SEQ ID NO: 45 - DNA (genomic), 14 bp; unknown-stranded, unknown
  hypothetical: YES
gcggaagtcaatgt

SEQ ID NO: 46 - DNA (genomic), 14 bp; unknown-stranded, unknown
  hypothetical: YES
agttggcatggtag

SEQ ID NO: 47 - DNA (genomic), 14 bp; unknown-stranded, unknown
  hypothetical: YES
gcagaagttggcat

SEQ ID NO: 48 - DNA (genomic), 14 bp; unknown-stranded, unknown
  hypothetical: YES
ctccaaatgtaggg

SEQ ID NO: 49 - DNA (genomic), 14 bp; unknown-stranded, unknown
  hypothetical: YES
accttgctgtactg

SEQ ID NO: 50 - DNA (genomic), 14 bp; unknown-stranded, unknown
  hypothetical: YES
tgctggttgtacag

SEQ ID NO: 51 - DNA (genomic), 14 bp; unknown-stranded, unknown
  hypothetical: YES
ggttatgctggttg

SEQ ID NO: 52 - DNA (genomic), 14 bp; unknown-stranded, unknown
  hypothetical: YES
gtagtacacgatgg

SEQ ID NO: 53 - DNA (genomic), 14 bp; unknown-stranded, unknown
  hypothetical: YES
cgtagtacacgatg

SEQ ID NO: 54 - DNA (genomic), 14 bp; unknown-stranded, unknown
  hypothetical: YES
cacgtagtacacga

SEQ ID NO: 55 - DNA (genomic), 14 bp; unknown-stranded, unknown
  hypothetical: YES
catgttggacagct

SEQ ID NO: 56 - DNA (genomic), 14 bp; unknown-stranded, unknown
  hypothetical: YES
gcacgatcatgttg

SEQ ID NO: 57 - DNA (genomic), 14 bp; unknown-stranded, unknown
  hypothetical: YES
cacacagtagtgca

SEQ ID NO: 58 - DNA (genomic), 14 bp; unknown-stranded, unknown
  hypothetical: YES
gatcagaaaagcgc

SEQ ID NO: 59 - DNA (genomic), 14 bp; unknown-stranded, unknown
  hypothetical: YES
accgtgaccagatg

SEQ ID NO: 60 - DNA (genomic), 14 bp; unknown-stranded, unknown
  hypothetical: YES
gtagacaggctgag

SEQ ID NO: 61 - DNA (genomic), 14 bp; unknown-stranded, unknown
  hypothetical: YES
tatcgagtgtgctg

SEQ ID NO: 62 - DNA (genomic), 14 bp; unknown-stranded, unknown
  hypothetical: YES
ttgcgcatgaactg

SEQ ID NO: 63 - DNA (genomic), 14 bp; unknown-stranded, unknown
  hypothetical: YES
ttgctcaggatctg

SEQ ID NO: 64 - DNA (genomic), 14 bp; unknown-stranded, unknown
  hypothetical: YES
actggtgagcttca

SEQ ID NO: 65 - DNA (genomic), 14 bp; unknown-stranded, unknown
  hypothetical: YES
atagtcttctgggg

SEQ ID NO: 66 - DNA (genomic), 14 bp; unknown-stranded, unknown
  hypothetical: YES
gctcaggatagtct

SEQ ID NO: 67 - DNA (genomic), 18 bp; unknown-stranded, unknown
  hypothetical: YES
tgtagatggaaatcacct

SEQ ID NO: 68 - DNA (genomic), 14 bp; unknown-stranded, unknown
  hypothetical: YES
tggtgctgttgtag

SEQ ID NO: 69 - DNA (genomic), 14 bp; unknown-stranded, unknown
  hypothetical: YES
ttctcctggagcaa

SEQ ID NO: 70 - DNA (genomic), 14 bp; unknown-stranded, unknown
  hypothetical: YES
tactcttcgtcgct

SEQ ID NO: 71 - DNA (genomic), 14 bp; unknown-stranded, unknown
  hypothetical: YES
cttggcgtagtact

SEQ ID NO: 72 - DNA (genomic), 18 bp; unknown-stranded, unknown
  hypothetical: YES
cggcatgtctattttgta

SEQ ID NO: 73 - DNA (genomic), 14 bp; unknown-stranded, unknown
  hypothetical: YES
ttttcggaggggaa

SEQ ID NO: 74 - DNA (genomic), 14 bp; unknown-stranded, unknown
  hypothetical: YES
cgggatggcatttt

SEQ ID NO: 75 - DNA (genomic), 14 bp; unknown-stranded, unknown
  hypothetical: YES
ctgtagaaagtggg

SEQ ID NO: 76 - DNA (genomic), 18 bp; unknown-stranded, unknown
  hypothetical: YES
acaattctgaagtagggt

SEQ ID NO: 77 - DNA (genomic), 18 bp; unknown-stranded, unknown
  hypothetical: YES
attgctgagacgtcaaat

SEQ ID NO: 78 - DNA (genomic), 14 bp; unknown-stranded, unknown
  hypothetical: YES
tctccattgctgag

SEQ ID NO: 79 - DNA (genomic), 18 bp; unknown-stranded, unknown
  hypothetical: YES
tcaccaaattggaagcat

SEQ ID NO: 80 - DNA (genomic), 14 bp; unknown-stranded, unknown
  hypothetical: YES
ctctgaactctgct

SEQ ID NO: 81 - DNA (genomic), 18 bp; unknown-stranded, unknown
  hypothetical: YES
aacgaaagactctgaact

SEQ ID NO: 82 - DNA (genomic), 14 bp; unknown-stranded, unknown
  hypothetical: YES
tgggttctgcaaac

SEQ ID NO: 83 - DNA (genomic), 14 bp; unknown-stranded, unknown
  hypothetical: YES
ctggcttttgggtt

SEQ ID NO: 84 - DNA (genomic), 14 bp; unknown-stranded, unknown
  hypothetical: YES
gttgttcaggcact

SEQ ID NO: 85 - DNA (genomic), 18 bp; unknown-stranded, unknown
  hypothetical: YES
tctgatatagctcaatcc

SEQ ID NO: 86 - DNA (genomic), 18 bp; unknown-stranded, unknown
  hypothetical: YES
tctttggacttgagaatc

SEQ ID NO: 87 - DNA (genomic), 14 bp; unknown-stranded, unknown
  hypothetical: YES
tgggttggagatgt

SEQ ID NO: 88 - DNA (genomic), 14 bp; unknown-stranded, unknown
  hypothetical: YES
tgctgtcgatgtag

SEQ ID NO: 89 - DNA (genomic), 16 bp; unknown-stranded, unknown
  hypothetical: YES
acaactttgctgtcga

SEQ ID NO: 90 - DNA (genomic), 14 bp; unknown-stranded, unknown
  hypothetical: YES
attcgccttctgct

SEQ ID NO: 91 - DNA (genomic), 14 bp; unknown-stranded, unknown
  hypothetical: YES
gaaggagagccatt

SEQ ID NO: 92 - DNA (genomic), 16 bp; unknown-stranded, unknown
  hypothetical: YES
tcagttacatcgaagg

SEQ ID NO: 93 - DNA (genomic), 18 bp; unknown-stranded, unknown
  hypothetical: YES
tgaagccattcatgaaca

SEQ ID NO: 94 - DNA (genomic), 16 bp; unknown-stranded, unknown
  hypothetical: YES
tcctgtctttatggtg

SEQ ID NO: 95 - DNA (genomic), 14 bp; unknown-stranded, unknown
  hypothetical: YES
aaatcccaggttcc

SEQ ID NO: 96 - DNA (genomic), 18 bp; unknown-stranded, unknown
  hypothetical: YES
ggacagtgtaagcttatt

SEQ ID NO: 97 - DNA (genomic), 16 bp; unknown-stranded, unknown
  hypothetical: YES
gtacaaaagtgcagca

SEQ ID NO: 98 - DNA (genomic), 18 bp; unknown-stranded, unknown
  hypothetical: YES
tagatggtacaaaagtgc

SEQ ID NO: 99 - DNA (genomic), 20 bp; unknown-stranded, unknown
  hypothetical: YES
cacttttatttgggatgatg

SEQ ID NO: 100 - DNA (genomic), 18 bp; unknown-stranded, unknown
  hypothetical: YES
gcaaatcttgcttctagt

SEQ ID NO: 101 - DNA (genomic), 14 bp; unknown-stranded, unknown
  hypothetical: YES
gtgccatcaatacc

SEQ ID NO: 102 - DNA (genomic), 14 bp; unknown-stranded, unknown
  hypothetical: YES
ggtatatgtggagg

SEQ ID NO: 103 - DNA (genomic), 14 bp; unknown-stranded, unknown
  hypothetical: YES
tctgatcaccactg

SEQ ID NO: 104 - DNA (genomic), 18 bp; unknown-stranded, unknown
  hypothetical: YES
tcctagtggactttatag

SEQ ID NO: 105 - DNA (genomic), 16 bp; unknown-stranded, unknown
  hypothetical: YES
tttttcctagtggact

SEQ ID NO: 106 - DNA (genomic), 14 bp; unknown-stranded, unknown
  hypothetical: YES
agatgtggggtctt

SEQ ID NO: 107 - DNA (genomic), 16 bp; unknown-stranded, unknown
  hypothetical: YES
caataacattagcagg

SEQ ID NO: 108 - DNA (genomic), 14 bp; unknown-stranded, unknown
  hypothetical: YES
aagtctgtaggagg

SEQ ID NO: 109 - DNA (genomic), 16 bp; unknown-stranded, unknown
  hypothetical: YES
tctgttgtgactcaag

SEQ ID NO: 110 - DNA (genomic), 14 bp; unknown-stranded, unknown
  hypothetical: YES
gttggtctgttgtg

SEQ ID NO: 111 - DNA (genomic), 14 bp; unknown-stranded, unknown
  hypothetical: YES
caaagcacgcttct

SEQ ID NO: 112 - DNA (genomic), 18 bp; unknown-stranded, unknown
  hypothetical: YES
tttctaaagcaataggcc

SEQ ID NO: 113 - DNA (genomic), 16 bp; unknown-stranded, unknown
  hypothetical: YES
gcaattatcctgcaca

SEQ ID NO: 114 - DNA (genomic), 14 bp; unknown-stranded, unknown
  hypothetical: YES
acgtaggcagcaat

SEQ ID NO: 115 - DNA (genomic), 18 bp; unknown-stranded, unknown
  hypothetical: YES
atcaatgtaaagtggacg

SEQ ID NO: 116 - DNA (genomic), 14 bp; unknown-stranded, unknown
  hypothetical: YES
ctagatccctcttg

SEQ ID NO: 117 - DNA (genomic), 14 bp; unknown-stranded, unknown
  hypothetical: YES
ccatttccacccta

SEQ ID NO: 118 - DNA (genomic), 14 bp; unknown-stranded, unknown
  hypothetical: YES
tgggttcgtgtatc

SEQ ID NO: 119 - DNA (genomic), 14 bp; unknown-stranded, unknown
  hypothetical: YES
tggcattgtaccct

SEQ ID NO: 120 - DNA (genomic), 14 bp; unknown-stranded, unknown
  hypothetical: YES
tccagcacagaagt

SEQ ID NO: 121 - DNA (genomic), 16 bp; unknown-stranded, unknown
  hypothetical: YES
ataaatacgggcatgc

SEQ ID NO: 122 - DNA (genomic), 14 bp; unknown-stranded, unknown
  hypothetical: YES
agtgtctgaactcc

SEQ ID NO: 123 - DNA (genomic), 14 bp; unknown-stranded, unknown
  hypothetical: YES
tgtgctgagtgtct

SEQ ID NO: 124 - DNA (genomic), 14 bp; unknown-stranded, unknown
  hypothetical: YES
ataagctcaggacc

SEQ ID NO: 125 - DNA (genomic), 14 bp; unknown-stranded, unknown
  hypothetical: YES
aggagaagcagatg

SEQ ID NO: 126 - DNA (genomic), 14 bp; unknown-stranded, unknown
  hypothetical: YES
agcaaggagaagca

SEQ ID NO: 127 - DNA (genomic), 14 bp; unknown-stranded, unknown
  hypothetical: YES
aatcttgggacacg

SEQ ID NO: 128 - DNA (genomic), 18 bp; unknown-stranded, unknown
  hypothetical: YES
tagagaatggttagaggt

SEQ ID NO: 129 - DNA (genomic), 18 bp; unknown-stranded, unknown
  hypothetical: YES
gttttgccaatgtagtag

SEQ ID NO: 130 - DNA (genomic), 14 bp; unknown-stranded, unknown
  hypothetical: YES
cttgggtgttttgc

SEQ ID NO: 131 - DNA (genomic), 16 bp; unknown-stranded, unknown
  hypothetical: YES
gcaagactttacaatc

SEQ ID NO: 132 - DNA (genomic), 18 bp; unknown-stranded, unknown
  hypothetical: YES
gcatttgcaagactttac

SEQ ID NO: 133 - DNA (genomic), 18 bp; unknown-stranded, unknown
  hypothetical: YES
tttagctgcatttgcaag

SEQ ID NO: 134 - DNA (genomic), 14 bp; unknown-stranded, unknown
  hypothetical: YES
gccacttttccaag

SEQ ID NO: 135 - DNA (genomic), 14 bp; unknown-stranded, unknown
  hypothetical: YES
ttggtcttgccact

SEQ ID NO: 136 - DNA (genomic), 14 bp; unknown-stranded, unknown
  hypothetical: YES
cagcacacagtagt

SEQ ID NO: 137 - DNA (genomic), 14 bp; unknown-stranded, unknown
  hypothetical: YES
cgatagtcttgcag